(19) United States
(12) Patent Application Publication
(10) Pub. No.: US 2022/0112523 A1
(43) Pub. Date: Apr. 14, 2022

(54) Triterpene Production

(71) Applicant: The Regents of the University of California, Oakland, CA (US)

(72) Inventors: Jay D. Keasling, Berkeley, CA (US); Yuzhong Liu, Berkeley, CA (US)

(73) Assignee: The Regents of the University of California, Oakland, CA (US)

(21) Appl. No.: 17/555,438

(22) Filed: Dec. 18, 2021

Related U.S. Application Data
(63) Continuation of application No. PCT/US20/36014, filed on Jun. 3, 2020.
(60) Provisional application No. 62/866,471, filed on Jun. 25, 2019.

(51) Int. Cl. C12P 7/00

(52) U.S. Cl. CPC C12P 7/00; C12Y 106/02004; C12Y 114/00; C12Y 504/99039

(57) ABSTRACT

An engineered microbial cell expressing a β-amyrin synthase, a cytochrome P450 reductase, a cytochrome P450 C28 oxidase, a cytochrome P450 C16 oxidase and a cytochrome C23 oxidase is used to make quillaic acid from β-amyrin.

### INTRODUCTION

Quillaic acid is a pentacyclic triterpenoid with hydroxy groups at positions 3 and 16, an aldehyde group at position 23 and a carboxylic acid at position 28 (FIG. 1), and has been demonstrated to have useful medicinal properties, e.g. Rodríguez-Díaz M, et al. Topical anti-inflammatory activity of quillaic acid from Quillaja saponaria Mol. and some derivatives. J Pharm Pharmacol. 2011 May; 63(5):718-24. Neither chemical synthesis nor the biosynthetic pathway of quillaic acid are known, and thus, there is an unmet need for quillaic acid production methods.

### SUMMARY OF THE INVENTION

The invention provides methods, composition and systems, such as engineered cells, for making β-amyrin oxidation products, such as quillaic acid, quillaic acid precursors and reduced and oxidized forms thereof.

In an aspect the invention provides a method of making an oxidized triterpene, comprising incubating or growing an engineered microbial cell expressing a β-amyrin synthase, a cytochrome P450 reductase, a cytochrome P450 C28 oxidase, a cytochrome P450 C16 oxidase and a cytochrome C23 oxidase under conditions wherein the C28 oxidase, the C16 oxidase and the C23 oxidize the C28, C16 and C23 carbons, respectively, of β-amyrin to carboxyl, hydroxyl, and formyl (aldehyde), respectively, to form the oxidized triterpenes.

In embodiments:

the microbial cell is a yeast cell, such as Saccharomyces cerevisiae, Pichia pastoris, or Hansenula polymorpha;

the microbial cell is an oleaginous yeast cell, such as Yarrowia lipolytica, Rhodosporidium toruloides or Lipomyces starkey;

the microbial cell is a bacterial cell, such as Escherichia coli, Bacillus subtilis, or Streptomyces spp.;

the microbial cell is engineered to express a plant β-amyrin synthase to divert the isoprenoid or native sterol biosynthetic pathway;

the cytochrome P450 reductase is selected from: Arabidopsis thaliana cytochrome P450 reductase (AtATR1) and Lotus japonicus cytochrome P450 reductase (LJCPR);

the cytochrome P450 C16 oxidase is selected from: CYP87D16 and CYP716Y1;

the cytochrome P450 C23 oxidase is selected from: CYP72A68 and CYP714E19;

the cytochrome P450 C28 oxidase is selected from: CYP716A1, CYP716A12, CYP716A15, CYP716A17, CYP716A44, CYP716A46, CYP716A52v2, CYP716A75, CYP716A78, CYP716A79, CYP716A80, CYP716A81, CYP716A83, CYP716A86, CYP716A154, CYP716A110, CYP716A140, CYP716A 141, CYP716A179, CYP716A252 and CYP716A253;

1, 2, 3 or all of the cytochrome P450 reductase, C28 oxidase, C16 oxidase and C23 oxidase are of plants, particularly of Arabidopsis thaliana, Lotus japonicus, Centella asiatica, Medicago truncatula, Bupleurum falcatum or Maesa lanceolate;

the cytochrome P450 reductase, C28 oxidase, C16 oxidase and C23 oxidase are independently of Arabidopsis thaliana, Lotus japonicus, Centella asiatica, Medicago truncatula, Bupleurum falcatum or Maesa lanceolate;

the C16 oxidase and C23 oxidase are: CYP72A68 (C23) and CYP716Y1 (C16);

the cytochrome P450 reductase, C28 oxidase, C16 oxidase and C23 oxidase are selected from combinations: Ljcpr+CYP72A68 (C23)+CYP716Y1 (C16).+CYP716A83 (C28); Ljcpr+CYP72A68 (C23)+CYP716Y1 (C16).+CYP716Al2 (C28); and Atrcpr+CYP72A68 (C23)+CYP716Y1 (C16).+CYP716Al2 (C28),

the oxidized triterpene is selected from quillaic acid, hederagenin, caulophylogenin, gypsogenin, gypsosenic acid and oxidized quillaic acid; and/or

the C23 carbon is oxidized to an acid, and optionally, later reducing the acid back to an aldehyde, or the C23 carbon is oxidized to an alcohol, and optionally later oxidizing the alcohol to an aldehyde, such as to form quaillaic acid.

In an aspect the invention provides an engineered microbial cell for making an oxidized triterpene, the cell expressing a β-amyrin synthase, a cytochrome P450 reductase, a cytochrome P450 C28 oxidase, a cytochrome P450 C16 oxidase and a cytochrome C23 oxidase, wherein the C28 oxidase, the C16 oxidase and the C23 oxidize the C28, C16 and C23 carbons, respectively, of β-amyrin to carboxyl, hydroxyl, and formyl (aldehyde), respectively, forming the oxidized triterpene.

The invention encompasses all combinations of the particular embodiments recited herein, as if each combination had been laboriously recited.

### BRIEF DESCRIPTION OF THE DRAWINGS

FIG. 1a. Biosynthetic pathway of 2,3-oxidosqualene production in Saccharomyces cerevisiae.

FIG. 1b. Downstream structural functionalization by expression of heterologous proteins to yield triterpenes, before the final glycosylation steps towards saponin synthesis. Solid arrows: corresponding enzymes have been identified; dotted arrows: enzymes are yet to be discovered.

FIG. 2. A simplified overview of quillaic acid biosynthesis pathway in the engineered S. cerevisiae. Alternating pathways for the synthesis of quillaic acid are shown with their corresponding enzymes listed at the upper right corner. Oxidation steps of C16, C23, and C28 are shown.

FIG. 3. In vivo combinatorial production of quillaic acid and other intermediates in yeast. Overlay of LC-MS chromatograms of standards (1: caulophyllogenin, 2: quillaic acid, 3: gypsogenic acid, 4: 16-hydroxyoleanolic acid, 5: hederagenin, 6: gypsogenin) in engineered strain expressing AtATR1, CYP72A68, CYP716Y1 and CYP716A12.

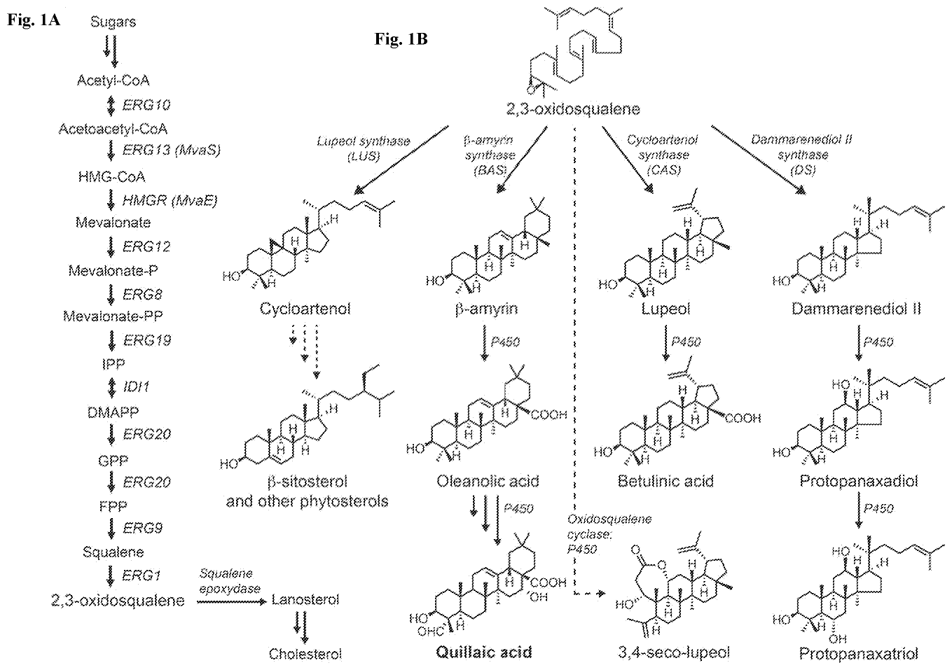

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We disclose the production of oxidized triterpenes by fermentation from engineered microbes, including yeast such as Saccharomyces cerevisiae using a combinatorial strategy of co-expressing heterologous proteins in the strain. Combinations of cytochrome P450 reductases and P450s from various plant origins were investigated. Since triterpenes constitute a large and structurally diverse class of natural products with various industrial and pharmaceutical applications, and P450-catalyzed structural modification is crucial for the diversification and functionalization of the triterpene scaffolds, our strategy provides a simple yet versatile platform to provide a renewable supply of triterpenes and their P450-functionalized products in the engineered yeast.

The combinatorial oxidase and terpene cyclase expression strategy together with a triterpene production strain also provides a platform to make other classes of naturally occurring triterpenes that are biologically active but difficult to extract and/or purify.

### EXAMPLE: QUILLAIC ACID BIOSYNTHESIS THROUGH COMBINATORIAL STUDIES OF HETEROLOGOUS PROTEINS

In this example, we present the production of quillaic acid, by fermentation from engineered Saccharomyces cerevisiae, using a combinatorial strategy of co-expressing heterologous proteins in a strain that has a high production yield of β-amyrin. Combinations of cytochrome P450 reductases and P450s from various plant origins are demonstrated. Because of enzymes' high specificity, P450s can selectively functionalize carbon positions of choice, forgoing the need to detour synthetic steps to ensure stereo- and chemoselectivity. The same strategy can also be utilized to make other classes of naturally occurring triterpenes, including oxidized β-amyrin products.

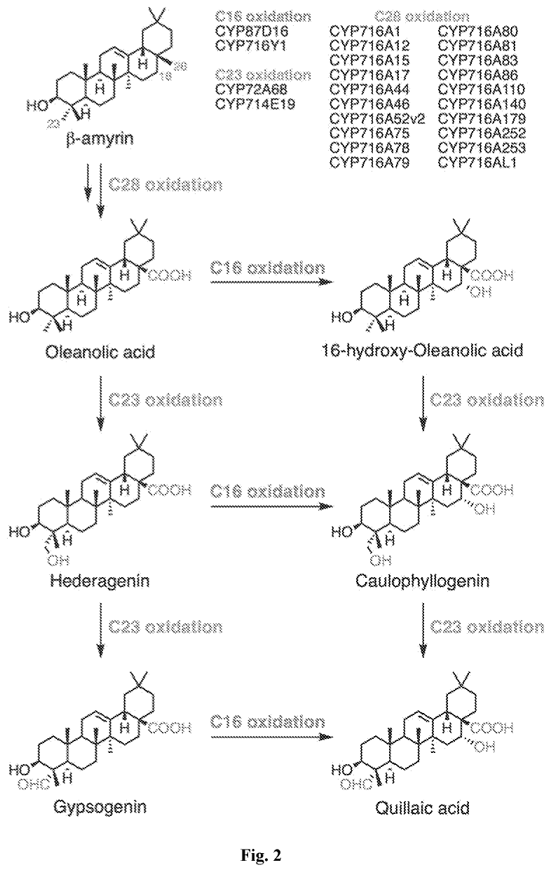

Since the natural quillaic acid biosynthetic pathway is still unknown, twenty-five P450s that have been characterized from Arabidopsis thaliana, Lotus japonicus, Centella asiatica, Medicago truncatula, Bupleurum falcatum, Maesa lanceolate and have been demonstrated for their enzymatic activity on β-amyrin as the substrate, were selected towards the combinatorial studies. Combinations of three P450s that include one enzyme for the functionalization on each carbon position (C16, C23 and C28) were expressed from high-copy number plasmids in an engineered yeast strain expressing a plant β-amyrin synthase to divert the native sterol biosynthetic pathway; see e.g., FIG. 2, and Kirby, Romanini, Paradise and Keasling, FEBS Journal 275 (8) April 2008, p1852-1859, “Engineering triterpene production in Saccharomyces cerevisiae β-amyrin synthase from Artemisia annua”. Quillaic acid production procedures were confirmed in terms of medium and sugar concertation, medium type, fermentation time, the use of additives, etc. Our results identify functional enzymes that can cooperatively convert β-amyrin to quillaic acid.

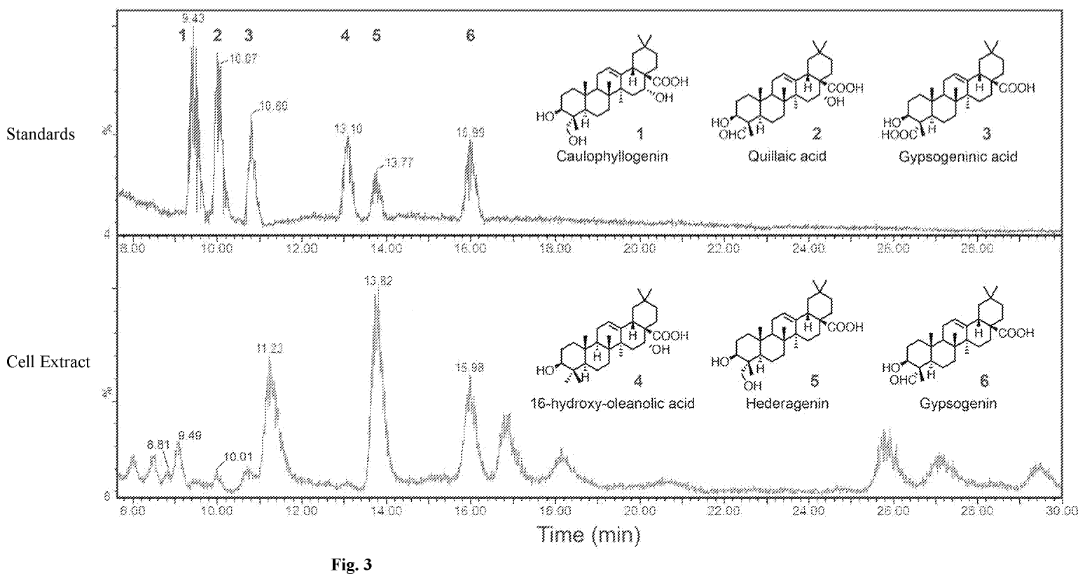

Liquid chromatography-mass spectrometry (LC-MS) was selected as the method of characterization to identify and purify quillaic acid produced from the yeast factory. The in vivo production level of the strain expressing AtATR1, CYP72A68, CYP716Y1 and CYP716A12 provides an example of the production from other different yeast constructs. In FIG. 3 the LC-MS chromatogram of extract from the yeast strain is compared with the authentic samples including quillaic acid (2) along with other oxidation intermediates: caulophyllogenin (1), gypsogenic acid (3), 16-hydroxyoleanolic acid (4), hederagenin (5), gypsogenin (6). While the accumulation of intermediates 3-6 was observed, the presence of the elution peak at 10.01 min corresponding to quillaic acid in the yeast extract unambiguously confirms its production in vivo.

We have also validated native P450 from Quillaja Saponaria (CYP716A224, QS28 oxidase; CYP714E52, QS23 oxidase) in Saccharomyces cerevisiae. CYP716A297, QS16 oxidase reactivity is also confirmed; these sequences are publicly-available.

1. A method of making an oxidized triterpene from β-amyrin, comprising incubating an engineered microbial cell expressing a β-amyrin synthase, a cytochrome P450 reductase, a cytochrome P450 C28 oxidase, a cytochrome P450 C16 oxidase and a cytochrome C23 oxidase under conditions wherein the C28 oxidase, the C16 oxidase and the C23 oxidize the C28, C16 and C23 carbons, respectively, of β-amyrin to carboxyl, hydroxyl, and formyl (aldehyde), respectively, to form the oxidized triterpene.

2. The method of claim 1, wherein the microbial cell is a yeast cell, selected from Saccharomyces cerevisiae, Pichia pastoris, and Hansenula polymorpha.

3. The method of claim 1, wherein the microbial cell is a yeast cell, that is Saccharomyces cerevisiae.

4. The method of claim 1, wherein the microbial cell is an oleaginous yeast cell selected from Yarrowia lipolytica, Rhodosporidium toruloides and Lipomyces starkey.

5. The method of claim 1, wherein the microbial cell is a bacterial cell selected from Escherichia coli, Bacillus subtilis, and Streptomyces spp.

6. The method of claim 1, wherein the microbial cell is engineered to express a plant β-amyrin synthase to divert the isoprenoid biosynthetic pathway.

7. The method of claim 1, wherein the cytochrome P450 reductase is selected from: Arabidopsis thaliana cytochrome P450 reductase (AtATR1) and Lotus japonicus cytochrome P450 reductase (LJCPR).

8. The method of claim 1, wherein the cytochrome P450 C16 oxidase is selected from: CYP87D16 and CYP716Y1.

9. The method of claim 1, wherein the cytochrome P450 C23 oxidase is selected from: CYP72A68 and CYP714E19.

10. The method of claim 1, wherein the cytochrome P450 C28 oxidase is selected from: CYP716A1, CYP716Al2, CYP716A15, CYP716A17, CYP716A44, CYP716A46, CYP716A52v2, CYP716A75, CYP716A78, CYP716A79, CYP716A80, CYP716A81, CYP716A83, CYP716A86, CYP716A154, CYP716A110, CYP716A140, CYP716A 141, CYP716A179, CYP716A252 and CYP716A253.

11. The method of claim 1, wherein the reductase, C28 oxidase, C16 oxidase and C23 oxidase are of plants independently selected from Arabidopsis thaliana, Lotus japonicus, Centella asiatica, Medicago truncatula, Bupleurum falcatum and Maesa lanceolate.

12. The method of claim 1, wherein the C16 oxidase and C23 oxidase are: CYP72A68 (C23) and CYP716Y1 (C16).

13. The method of claim 1, wherein the reductase, C28 oxidase, C16 oxidase and C23 oxidase are selected from combinations:
Ljcpr+CYP72A68 (C23)+CYP716Y1 (C16)+CYP716A83 (C28);
Ljcpr+CYP72A68 (C23)+CYP716Y1 (C16)+CYP716A12 (C28); and
Atrcpr+CYP72A68 (C23)+CYP716Y1 (C16)+CYP716A12 (C28).

14. The method of claim 2, wherein the reductase, C28 oxidase, C16 oxidase and C23 oxidase are selected from combinations:
Ljcpr+CYP72A68 (C23)+CYP716Y1 (C16)+CYP716A83 (C28);
Ljcpr+CYP72A68 (C23)+CYP716Y1 (C16)+CYP716A12 (C28); and
Atrcpr+CYP72A68 (C23)+CYP716Y1 (C16)+CYP716A12 (C28).

15. The method of claim 3, wherein the reductase, C28 oxidase, C16 oxidase and C23 oxidase are selected from combinations:
Ljcpr+CYP72A68 (C23)+CYP716Y1 (C16)+CYP716A83 (C28);
Ljcpr+CYP72A68 (C23)+CYP716Y1 (C16)+CYP716A12 (C28); and
Atrcpr+CYP72A68 (C23)+CYP716Y1 (C16)+CYP716A12 (C28).

16. The method of claim 1, wherein the oxidized triterpene is selected from quillaic acid, hederagenin, caulophylogenin, gypsogenin, gypsosenic acid and oxidized quillaic acid.

17. The method of claim 15, wherein the oxidized triterpene is selected from quillaic acid, hederagenin, caulophylogenin, gypsogenin, gypsosenic acid and oxidized quillaic acid.

18. The method of claim 1, wherein the C23 carbon is oxidized to an acid, or the C23 carbon is oxidized to an alcohol.

19. The method of claim 1, wherein the C23 carbon is oxidized to an acid, and the method further comprises reducing the acid back to an aldehyde, or the C23 carbon is oxidized to an alcohol, and the method further comprises oxidizing the alcohol back to an aldehyde.

20. An engineered microbial cell for making an oxidized triterpene, the cell expressing a β-amyrin synthase, a cytochrome P450 reductase, a cytochrome P450 C28 oxidase, a cytochrome P450 C16 oxidase and a cytochrome C23 oxidase, wherein the C28 oxidase, the C16 oxidase and the C23 oxidize the C28, C16 and C23 carbons, respectively, of β-amyrin to carboxyl, hydroxyl, and formyl (aldehyde), respectively, forming the oxidized triterpene.
